SEQUENCE LISTING (15 sequences)

SEQ ID NO: 1 - DNA, 33 bp, Artificial Sequence
  primer OYE2-1
cggtccagatatagaataaatcatcatattaag

SEQ ID NO: 2 - DNA, 29 bp, Artificial Sequence
  primer OYE2-2
gaaatggtgctacaaagtacggttaacac

SEQ ID NO: 3 - DNA, 25 bp, Artificial Sequence
  primer OYE2-3
ttagaagaattcatgccatttgtta

SEQ ID NO: 4 - DNA, 25 bp, Artificial Sequence
  primer OYE2-4
agatttctgcagttaatttttgtcc

SEQ ID NO: 5 - DNA, 21 bp, Artificial Sequence
  primer PKK223-3(+)
gacatcataacggttctggca

SEQ ID NO: 6 - DNA, 21 bp, Artificial Sequence
  primer PKK223-3(-)
ttatcagaccgcttctgcgtt

SEQ ID NO: 7 - DNA, 20 bp, Artificial Sequence
  primer OYE2-5
ggtatctggtccgaagaaca

SEQ ID NO: 8 - DNA, 21 bp, Artificial Sequence
  primer OYE2-6
gacacgaggttcaactagatg

SEQ ID NO: 9 - DNA, 30 bp, Artificial Sequence
  primer OYE3-1
gtacgtacttgatatatacaacaactgtag

SEQ ID NO: 10 - DNA, 28 bp, Artificial Sequence
  primer OYE3-2
gctgccctatataaacaaagatcgagtc

SEQ ID NO: 11 - DNA, 25 bp, Artificial Sequence
  primer OYE3-5
ttagaacaattgatgccatttgtaa

SEQ ID NO: 12 - DNA, 24 bp, Artificial Sequence
  primer OYE3-4
agatttctgcagtcagttcttgtt

SEQ ID NO: 13 - DNA, 21 bp, Artificial Sequence
  primer PKK223-3(+)
gacatcataacggttctggca

SEQ ID NO: 14 - DNA, 21 bp, Artificial Sequence
  primer PKK223-3(-)
ttatcagaccgcttctgcgtt

SEQ ID NO: 15 - DNA, 20 bp, Artificial Sequence
  primer OYE3-6
gactgtgcatctgacagagt

(19) United States
(12) Patent Application Publication
(10) Pub. No.: US 2005/0244941 A1
(43) Pub. Date: Nov. 3, 2005

(54) Process for producing levodione

(76) Inventors: Sakayu Shimizu, Kyoto-fu (JP); Masaru Wada, Sapporo (JP)

Correspondence Address: Stephen M Haracz;Bryan Cave, 1290 Avenue of the Americas, New York, NY, 10104-3300, US

(21) Appl. No.: 10/505,314

(22) PCT Filed: Feb. 15, 2003
(86) PCT No.: PCT/EP03/01537

§ 371 Date: Apr. 11, 2005

(30) Foreign Application Priority Data
Feb. 22, 2002 (EP) 020 03 968.1

(51) Int. Cl. C12P 7/26; C12N 1/18; C12N 15/74

(52) U.S. Cl. 435/148; 435/254.21; 435/483

(57) ABSTRACT

An enone reductase characterized by a molecular mass of 61,300±5,000 Da; NADPH and NADH as co-factor; a temperature optimum of 55-60° C. at pH 7.4; a pH optimum of 4.5-8.5 and a substrate specificity on α,β-unsaturated ketones, especially derived from a yeast and a process for the preparation of levodione from ketoisophorane.

The present invention relates to a process for producing (6R)-2,2,6-trimethyl cyclohexane-1,4-dione (hereinafter referred to as levodione) from 2,6,6-trimethly-2-cyclohexene-1,4-dione (hereinafter referred to as ketoisophorone) by reduced nicotinamide adenine dinucleotide phosphate (herein after referred to as NADPH) dehydrogenase. Levodione is an important intermediate in the synthesis of carotenoids, e.g. zeaxanthin.

A microbiological process of producing levodione from ketoisophorone has been known, see e.g. U.S. Pat. No. 4,156,100.

Unexpectedly, we now have found that levodione can be formed from ketoisophorone by using a NADPH dehydrogenase as a catalyst. The NADPH dehydrogenases for use as catalysts in the process of the present invention are generally known as old yellow enzyme (OYE) and defined by the enzyme class E.C. 1.6.99 according to the Enzyme Nomenclature provided by the Nomenclature Committee of the International Union of Biochemistry and Molecular Biology (Academic Press, 1992).

The present invention is related to a process for producing levodione from ketoisophorone which comprises contacting ketoisophorone with NADPH dehydrogenase in the presence of NADH or NADPH in an aqueous medium, and isolating the resulted levodione from the reaction mixture.

As used herein, the term “NADPH dehydrogenase” encompasses proteins capable of catalyzing the enzymatic reaction of ketoisophorone to levodione in the presence of NADH or NADPH, such as the old yellow enzyme (OYE) classified as EC 1.6.99 according to the Enzyme Nomenclature provided by the Nomenclature Committee of the International Union of Biochemistry and Molecular Biology.

Particularly, the present invention is related to a process for producing levodione from ketoisophorone wherein the NADPH dehydrogenase catalyzing said reaction is OYE defined by the enzyme class EC 1.6.99.

OYE for use as catalyst in the process of the present invention is obtainable or may be isolated from any appropriate microorganisms suitable for the production of said enzyme, including but not limited to the genera Saccharomyces, Zygosaccharomyces, Candida, Gluconobacter, Beneckea, or Vibrio. The above mentioned microorganisms also include synonyms or basonyms of such microorganisms having the same physico-chemical properties, as defined by the International Code of Nomenclature of Prokaryotes.

A transformed microorganism, such as Escherichia coli, expressing NADPH dehydrogenase can also be used as a starting microorganism.

In one embodiment of the present invention, the microorganism suitable for the production of NADPH dehydrogenase such as OYE is Saccharomyces cerevisiae, preferably Saccharomyces cerevisiae S288C (ATCC 204508) publicly available from the American Type Culture Collection, 10801 University Boulevard Manassas, Va. 20100-2209, U.S.A. The well known purification process can be used for the preparation of the enzyme (cf. Abramovitz, A. S., & Massey. V., J. Biol. Chem. 251,5321-5326,1976). Functional equivalents, subcultures, mutants and variants of said microorganism can also be used in the present invention.

In one embodiment of the present invention, a commercially available OYE can be used for the catalytic cleavage of ketoisophorone into levodione, for instance, NADPH-FMN Oxidoreductase® (Sigma, U.S.A.).

The OYE used preferably for the present invention is composed of two subunits, OYE2 and OYE3 having a molecular weight of 45.0 kDa and 44.9 kDa, respectively. Surprisingly, it was found that OYE2 and OYE3 not only can use NADPH, but also NADH as a co-factor for catalyzing the reaction of the present invention.

Thus, it is a further aspect of the present invention to provide a process wherein the OYE used for the catalytic reaction of ketoisophorone into levodione is encoded by the oye2 or oye3 gene derived from Saccharomyces cerevisiae S288C (ATCC 204508).

OYE of the present invention catalyzes the reduction of ketoisophorone to levodione in the presence of a co-factor according to the following formula:
Ketoisophorone+NADH (NADPH) [symbol] Levodione+NAD (NADP)

For example, the standard enzyme reaction is performed as follows: the basal reaction mixture (100 μL of 1 M Tris-HCl buffer, pH 8.0, 100 μl of 80 mM NADH, 100 μl of 0.13 M ketoisophorone, and water to fill up to 1 ml of total volume), is supplemented by 5 μl of the enzyme solution, and is incubated at 20 to 40° C. for 5-300 min, preferably at 25° C. for 30 min. The reaction mixture is extracted by 1 ml of an organic solvent such as ethyl acetate, n-hexane, toluene, or n-butyl acetate to recover the levodione into the organic solvent layer. The extract is analyzed by known methods such as gas chromatography, high performance liquid chromatography, thin layer chromatography or paper chromatography, or the like. In case of the gas chromatography, the following conditions can be applied as one embodiment:
Column: ULBON HR-20M (Shinwa, Japan) 0.25 mmφ×30 m
Column temperature: 160° C. (constant)
Injector temperature: 250° C.
Carrier gas: He (ca. 1 ml/min)

The reaction can be conducted at pH values of from about 4.5 to about 8.5 in the presence of NADH or NADPH in a solvent, such as Tris-HCl buffer, phosphate buffer and the like. Preferably, the pH is between 5.0 and 8.0.

The present invention relates to a process for the production of levodione from ketoisophorone by the help of OYE, wherein the reaction is carried out at pH values of from 4.5 to 8.5 and at a temperature in the range of from 20 to 40° C. Preferably, the reaction is carried out at pH values of from 5.0 to 8.0 and at a temperature in the range of from 25 to 35° C.

The genes encoding the proteins OYE2 and OYE3 of the present invention can be cloned based on the genomic sequence information of the originating microorganism, e.g. Saccharomyces cerevisiae, and can be overexpressed in an appropriate host organism such as Escherichia coli. The recombinant microorganism, such as Escherichia coli, expressing NADPH dehydrogenase can be prepared by well known recombinant technologies (cf. Molecular cloning: a Laboratory Manual, 2nd Edition/Cold Spring Harbor Laboratory Press, 1989).

Thus, the present invention concerns a process for producing levodione from ketoisophorone which comprises contacting ketoisophorone with a recombinant microorganism expressing NADPH dehydrogenase or cell-free extract thereof in the presence of NADH or NADPH in an aqueous medium, and isolating the obtained levodione from the reaction mixture. Preferably, the recombinant microorganism is Escherichia coli.

The NADPH dehydrogenase expressed by the recombinant microorganism is OYE defined by the enzyme class EC 1.6.99 as in another aspect of the present invention.

Particularly, the OYE used for the expression in the recombinant microorganism is derivable from a microorganism which is selected from the group consisting of Saccharomyces, Zygosaccharomyces, Candida, Gluconobacter, Beneckea and Vibrio or functional equivalents, subcultures, mutants and variants thereof. Preferably, the OYE is derived from Saccharomyces cerevisiae, more preferably Saccharomyces cerevisiae S288C (ATCC 204508). Most preferred is OYE encoded by the oye2 or oye3 gene from Saccharomyces cerevisiae S288C (ATCC 204508).

The recombinant microorganism, such as e.g. Escherichia coli, expressing NADPH dehydrogenase, may be cultured in a nutrient medium containing saccharides such as glucose or sucrose, alcohols such as ethanol or glycerol, fatty acids such as oleic acid, stearic acid or esters thereof, or oils such as rapeseed oil or soybean oil as carbon sources; ammonium sulfate, sodium nitrate, peptone, amino acids, corn steep liquor, bran, yeast extract and the like as nitrogen sources; magnesium sulfate, sodium chloride, calcium carbonate, potassium monohydrogen phosphate, potassium dihydrogen phosphate and the like as inorganic salt sources; and malt extract, meat extract and the like as other nutrient sources. The cultivation can be carried out aerobically, normally with a cultivation period of from 1 to 7 days in a medium pH of from 3.0 to 9.0 and at a temperature in the range of from 10 to 40° C. Preferably, the cultivation is carried out at the medium pH of from 5.0 and 8.0, and the cultivation temperature of from 25 to 35° C. for from 2 to 4 days.

OYE is useful as a catalyst for the production of levodione from ketoisophorone. In one embodiment, the reaction for producing levodione from ketoisophorone using a recombinant microorganism can be conducted at pH values of from about 4.5 to about 8.5 in the presence of NADH or NADPH in a solvent, such as Tris-HCl buffer, phosphate buffer and the like. In a preferred embodiment, the reaction is carried out at a pH between 5.0 and 8.0.

A preferred temperature range for carrying out the reaction is from 20 to 40° C. When the pH and the temperature are set at 5.0 to 8.0 and 25 to 35° C., respectively, the reaction usually produces the best results.

In a further embodiment, the reaction for producing levodione using a recombinant microorganism is carried out at pH values of from 4.5 to 8.5 and at a temperature in the range of from 20 to 40° C. Preferably, the reaction is carried out at pH values of from 5.0 to 8.0 and at a temperature in the range of from 25 to 35° C.

The concentration of ketoisophorone in a solvent can vary depending on other reaction conditions, but, in general, is between 1 mM and 2 M, preferably between 10 mM and 100 mM.

In the reaction, OYE may also be used in an immobilized state with an appropriate carrier. Any means of immobilizing enzymes generally known in the art may be used. For instance, the enzyme may be bound directly to a membrane, granules or the like of a resin having one or more functional groups or it may be bound to the resin through bridging compounds having one or more functional groups, e.g. glutaraldehyde.

After the reaction, levodione may be recovered from the reaction mixture by extraction with an organic solvent which is non-miscible with water and which readily solubilizes levodione, such as ethyl acetate, n-hexane, toluene or n-butyl acetate. Further purification of levodione can be effected by concentrating the extract to directly crystallize levodione or by the combination of various kinds of chromatography, for example, thin layer chromatography, adsorption chromatography, ion-exchange chromatography, gel filtration chromatography or high performance liquid chromatography.

The following Examples further illustrate the present invention.

### EXAMPLE 1

Cloning of oye2 and oye3 Genes from Genomic DNA of Saccharomyces cerevisiae

Genomic DNA of Saccharomyces cerevisiae S288C (ATCC 204508) was prepared using the potassium acetate method. Using the prepared genomic DNA as template, the gene fragments of oye2 and oye3 were obtained by two-step PCR method using a thermal cycler (Perkin Elmer 2400, U.S.A.). The PCR mixture (0.02 ml) contained 5 pmol of each primer, 0.312 mM of each dNTP, and 2.5 U of Pyrobest DNA polymerase (Takara Shuzo Co. LTD/Kyoto, Japan). 100 ng of the genomic DNA of Saccharomyces cerevisiae S288C (ATCC 204508) was used as the template for the first PCR reaction. The mixture after the reaction was diluted 1:20 in water, and used as the template for the second PCR reaction. For the first PCR, a cycle of 10 sec. at 98° C., 30 sec. at 55° C. and 90 sec. at 72° C. was repeated for 25 times. For the second PCR, a cycle of 10 sec. at 98° C., 30 sec. at 51° C. and 90 sec. at 72° C. was repeated for 25 times.

To done the oye2 gene, the first PCR reaction was performed with primers, OYE2-1 (5′-CGGTCCAGATATAGAATAAATCATCATATTAAG-3′) (SEQ ID NO: 1), and OYE2-2 (5′-GAAATGGTGCTACAAAGTACGGTTAACAC-3′) (SEQ ID NO: 2). By this reaction, DNA fragment containing the oye2 gene (1250 bp) was amplified. The second PCR was performed with primers, OYE2-3 (5′-TTAGAAGAATTCATGCCATTTGTTA-3′) (SEQ ID NO: 3) and OYE2-4 (5′-AGATTTCTGCAGTTAATTTTTGTCC-3′) (SEQ ID NO: 4).

By this reaction, DNA fragment containing just the ORF of the oye2 gene (1200 bp) was amplified. This amplified oye2 gene was treated with EcoRI and PstI, and ligated with a vector, pKK223-3 (Amersham Bioscience/Buckinghamshire, England) that was predigested with EcoRI and PstI to construct a plasmid, pKK223-3/OYE2. E. coli DH5a was transformed with the ligation mixture, and several clones were selected for sequence analysis. The sequence of the cloned oye2 gene of each candidate done was determined by using the “Thermo Sequenase II dye terminator cycle sequencing kit” (Amersham Bioscience/Buckinghamshire, England) and an automatic sequence analyzer (ABI prism 377). Primers used for the sequence analysis were as follows:

| | |
|---|---|
| PKK223-3(+) | |
| (5′-GACATCATAACGGTTCTGGCA-3′) | (SEQ ID NO: 5) |
| PKK223-3(−) | |
| (5′-TTATCAGACCGCTTCTGCGTT-3′) | (SEQ ID NO: 6) |
| OYE2-5 | |
| (5′-GGTATCTGGTCCGAAGAACA-3′) | (SEQ ID NO: 7) |
| OYE2-6 | |
| (5′-GACACGAGGTTCAACTAGATG-3′). | (SEQ ID NO: 8) |

One of the clones that showed completely the same sequence as the known oye2 sequence of Saccharomyces cerevisiae S288C (ATCC 204508) was selected for further experiments. To clone the oye3 gene, the first PCR reaction was performed with primers OYE3-1 (5′-GTACGTACTTGATATATACAACAACTGTAG-3′) (SEQ ID NO: 9) and OYE3-2 (5′-GCTGCCCTATATAAACAAAGATCGAGTC-3′) (SEQ ID NO: 10).

By this reaction, DNA fragment containing the oye3 gene (1250 bp) was amplified. The second PCR was performed with the following primers:

| | |
|---|---|
| OYE3-5 | |
| (5′-TTAGAACAATTGATGCCATTTGTAA-3′) | (SEQ ID NO: 11) |
| OYE3-4 | |
| (5′-AGATTTCTGCAGTCAGTTCTTGTT-3′). | (SEQ ID NO: 12) |

By this reaction, DNA fragment containing just the ORF of oye3 gene (1200 bp) was amplified. This amplified oye3 gene was treated with MfeI and PstI, and ligated with a vector, pKK223-3 (Amersham Bioscience/Buckinghamshire, England) that was predigested with EcoRI and PstI to construct a plasmid, pKK223-3/OYE3. E. coli DH5α was transformed with the ligation mixture, and several clones were selected for sequence analysis. The sequence of the cloned oye3 gene of each candidate clone was determined by using “Thermo Sequenase II dye terminator cycle sequencing kit” (Amersham Bioscience/Buckinghamshire, England) and an automatic sequence analyzer (ABI prism 377). Primers used for the sequence analysis were as follows:

| | |
|---|---|
| PKK223-3(+) | |
| (5′-GACATCATAACGGTTCTGGCA-3′) | (SEQ ID NO: 13) |
| PKK223-3(−) | |
| (5′-TTATCAGACCGCTTCTGCGTT-3′) | (SEQ ID NO: 14) |
| OYE3-6 | |
| (5′-GACTGTGCATCTGACAGAGT-3′). | (SEQ ID NO: 15) |

One of the clones that showed completely the same sequence as the known oye3 sequence of Saccharomyces cerevisiae S288C (ATCC 204508) was selected for further experiments.

### EXAMPLE 2

Levodione Production Using the Cell-Free Extract of E. coli Strain Having the oye2 or oye3 Gene of Saccharomyces cerevisiae

The plasmids, pKK223-3/OYE2 and pKK223-3/OYE3, which were constructed in the Example 1 and which comprise the complete DNA sequence of oye2 and oye3, respectively, were introduced into E. coli JM109, and the recombinant strains, JM109[pKK223-3/OYE2] and JM109[pKK223-3/OYE3] were obtained.

The strain JM109[pKK223-3] was also prepared as a control. Each of these strains was inoculated into the M9 minimum medium (2×500 ml in 2L-Sakaguchi flask) containing 0.05 mg/ml of ampicillin and 2% (w/v) of casamino acids (Difco Laboratories, U.S.A.) and cultivated at 37° C. When the optical density at 610 nm reached 0.4, IPTG (isopropyl β-D-thiogalactopyranoside) was added to the medium to make the concentration 0.01 mM and cultivation was continued for further 8-10 hours. Then the bacterial cells were collected by centrifugation. Approximately 10 g of wet cells were obtained from 1 liter of the broth. A fraction (0.7 g) of the cells was resuspended into 1.4 ml of the buffer consisting of 40 mM Tris-HCl (pH 8.0), 10 mM MgCl₂, 10 mM dithiothreitol (DTT), 200 mM KCl and 1 mM phenylmethylsulfonyl fluoride (PMSF), and the cells were disrupted with an ultrasonic oscillator for 15 min. After centrifugation, the resulting supernatant was used as a cell-free extract for the levodione production as follows. Each of the cell-free extract obtained from the cells of JM109[pKK223-3/OYE2], JM109[pKK223-3/OYE3] or JM109[pKK223-3] containing 2 mg protein was used in 1 ml of the reaction mixture consisting of 25 mM Tris-HCl (pH 8.0), 66 mM NADH or 55 mM NADPH, and 13 mM of ketoisophorone. The reaction was carried out at 25° C. for 30 minutes. The reaction mixture was extracted by 1 ml of ethylacetate to recover the levodione into ethylacetate layer. The extract was analyzed by gas chromatography [column: ULBON HR-20M (Shinwa, Japan) 0.25 mmφ×30 m, column temperature: 160° C. (constant), injector temperature: 250° C., carrier gas: He (ca. 1 ml/min)]. The results are shown in Table 1.

TABLE 1

| | | Yield of Levodione (% of |
|---|---|---|
| Clone | Co-factor | ketoisophorone used) |
| JM109[pKK223-3/OYE2] | NADH | 60 |
| JM109[pKK223-3/OYE2] | NADPH | 34 |
| JM109[pKK223-3/OYE3] | NADH | 12 |
| JM109[pKK223-3/OYE3] | NADPH | 12 |
| JM109[pKK223-3] (control) | NADH | 8.5 |
| JM109[pKK223-3] (control) | NADPH | 9.9 |

In another experiment using the combination of the cell-free extract of JM109[pKK223-3/OYE2] with the NADH-recycling system, the yield of levodione reached 95%. In this case, the cell-free extract of JM109[pKK223-3/OYE2] containing 30 mg protein was used in 25 ml of the reaction mixture consisting of 250 mM Tris-HCl (pH 8.0), 0.31 mM NAD⁺, 220 mM D-glucose, 12.5 units/ml glucose dehydrogenase and 65 mM of ketoisophorone. The reaction was carried out at room temperature for 90 min. The pH of the reaction mixture was controlled to be higher than 7.0 using 7% ammonium solution. As a result, 9.5 g/l of levodione (95% conversion of ketoisophorone used) was produced. Optical purity of the product was analyzed to be 94.6% (enantiomeric excess; e.e.) by gas chromatography using a chiral capillary column, BGB-176 (BGB Analytik AG, Switzerland).

### EXAMPLE 3

Levodione Production Using the Cells of E. coli Strain Having the oye Gene of Saccharomyces cerevisiae

Each of the strains, JM109[pKK223-3/OYE2] and JM109[pKK223-3], obtained in Example 2 was inoculated into the M9 minimum medium (5 ml in tube) containing 0.05 mg/ml of ampicillin and 2% (w/v) of casamino acids (Difco Laboratories, U.S.A.) and cultivated at 37° C. When the optical density at 610 nm reached 0.4, IPTG was added to the medium to make the concentration 0.01 mM and cultivation was continued for further 8-10 hours. Then the bacterial cells were collected by centrifugation, and resuspended into 2 ml of 100 mM potassium phosphate buffer (pH 7.0). This suspension was divided into two portions (1 ml each), and the reaction was started by adding 33 mM (final concentration, hereinafter abbreviated as f.c.) of ketoisophorone and 280 mM (f.c.) of D-glucose with or without 0.37 mM (f.c.) of NAD⁺, 15 units/ml (f.c.) of glucose dehydrogenase. The reaction was carried out at 30° C. overnight. The reaction mixture was extracted by ethylacetate to recover the levodione into ethylacetate layer. The extract was analyzed by gas chromatography as described in Example 1. The results are shown in Table 2.

TABLE 2

| | Glucose de- | Yield of Levodione | Optical |
|---|---|---|---|
| | hydrogenase | (% of ketoiso- | purity |
| Clone | & NAD⁺ | phorone used) | (% e.e.) |
| JM109[pKK223-3/OYE2] | − | 65 | 59 |
| JM109[pKK223-3/OYE2] | + | 65 | 64 |
| JM109[pKK223-3] | − | <1 | — |
| (control) | | | |
| JM109[pKK223-3] | + | <1 | — |
| (control) | | | |

1. A process for producing levodione from ketoisophorone which comprises contacting ketoisophorone with NADPH dehydrogenase in the presence of NADH or NADPH in an aqueous medium, and isolating the resulted levodione from the reaction mixture.

2. The process according to claim 1, wherein the NADPH dehydrogenase is old yellow enzyme defined by the enzyme class EC 1.6.99.

3. The process according to claim 1, wherein the enzyme is obtainable from a microorganism suitable for the production of the NADPH dehydrogenase.

4. The process according to claim 3, wherein the microorganism is selected from the group of genera consisting of Saccharomyces, Zygosaccharomyces, Candida, Gluconobacter, Beneckea, and Vibrio.

5. The process according to claim 3, wherein the microorganism is Saccharomyces cerevisiae.

6. The process according to claim 3, wherein the microorganism is Saccharomyces cerevisiae S288C (ATCC 204508), a functional equivalent, subculture, mutant or variant thereof.

7. The process according to claim 1, wherein the NADPH dehydrogenase is old yellow enzyme encoded by the oye2 or oye3 gene derived from Saccharomyces cerevisiae S288C (ATCC 204508).

8. The process according to claim 1, wherein the reaction is carried out at pH values of from 4.5 to 8.5 and at a temperature in the range of from 20 to 40° C.

9. The process according to claim 1, wherein the reaction is carried out at pH values of from 5.0 to 8.0 and at a temperature in the range of from 25 to 35° C.

10. A process for producing levodione from ketoisophorone which comprises contacting ketoisophorone with a transformed microorganism expressing NADPH dehydrogenase or a cell-free extract thereof in the presence of NADH or NADPH in an aqueous medium, and isolating the obtained levodione from the reaction mixture.

11. The process according to claim 10, wherein the transformed microorganism is Escherichia coli.

12. The process according to claim 10, wherein the NADPH dehydrogenase is old yellow enzyme defined by the enzyme class EC 1.6.99.

13. The process according to claim 10, wherein the enzyme expressed by the transformed microorganism is derivable from a microorganism selected from the group consisting of the genera Saccharomyces, Zygosaccharomyces, Candida, Gluconobacter, Beneckea, and Vibrio.

14. The process according to claim 10, wherein the enzyme expressed by the transformed microorganism is derived from Saccharomyces cerevisiae.

15. The process according to claim 10, wherein the NADPH dehydrogenase expressed by the transformed microorganism is old yellow enzyme encoded by the oye2 or oye3 gene derived from Saccharomyces cerevisiae S288C (ATCC 204508).

16. The process according to claim 10, wherein the reaction is carried out at pH values of from 4.5 to 8.5 and at a temperature in the range of from 20 to 40° C.

17. The process according to claim 10, wherein the reaction is carried out at pH values of from 5.0 to 8.0 and at a temperature in the range of from 25 to 35° C.

18. (canceled)

19. The process according to claim 2, wherein the enzyme is obtainable from a microorganism suitable for the production of the old yellow enzyme.

20. The process according to claim 11, wherein the NADPH dehydrogenase is old yellow enzyme defined by the enzyme class EC 1.6.99.

21. The process according to claim 14, wherein the microorganism is Saccharomyces cerevisiae S288C (ATCC 204508).
